(19) United States
(12) Patent Application Publication
(10) Pub. No.: US 2012/0064177 A1
(43) Pub. Date: Mar. 15, 2012

(54) METHOD OF PREPARING AN INTRANASAL COMPOSITION AND INTRANASAL COMPOSITIONS OBTAINABLE THEREBY

(76) Inventor: Paul Carpenter, Newtown, CT (US)

(21) Appl. No.: 12/879,090

(22) Filed: Sep. 10, 2010

(51) Int. Cl. A61K 33/14; A61K 36/61; A61P 29/00; A61K 31/375; A61P 37/00; A61K 31/165; A61K 36/886

(52) U.S. Cl. 424/665; 514/627; 424/742; 424/744; 514/474

(57) ABSTRACT

The present invention relates to a method of preparing an intranasal composition including from about 0.0010-0.0040% by weight capsaicin, from about 0.50-0.90% by weight hydroxypropyl methylcellulose, from about 0.010-0.080% by weight n-alkyl dimethyl benzyl ammonium chloride (Quat), and various moisturizing and irrigation agents, wherein the method includes dissolving the above components in water to form an aqueous solution; and filtering the aqueous solution to form an intranasal composition. The present invention further relates to an intranasal drug delivery composition, which includes from about 0.0010-0.0040% by weight capsaicin, from about 0.50-0.90% by weight hydroxypropyl methylcellulose, from about 0.010-0.080% by weight n-alkyl dimethyl benzyl ammonium chloride (Quat), and various moisturizing and irrigation agents. The present invention further relates to a method to increase tolerability and reduce burning from capsaicin when applied to nasal mucosa in the form of an intranasal composition, which includes combining capsaicin with the materials as noted above.

### FIELD OF THE INVENTION

The present invention relates to a method of preparing an intranasal composition including hydroxypropyl methylcellulose, and intranasal compositions capable of being produced thereby.

### BACKGROUND OF THE INVENTION

Pharmaceutical compositions have been used for the treatment of rhinitis, sinusitis, hay fever, colds and other inflammatory conditions of the nasal cavities for some time. Often, the pharmaceutical compositions are administered nasally in the form of drops or sprays. The success of treatment of a particular condition may not only depend upon the nature of the pharmaceutically active ingredient in the pharmaceutical composition, but may also depend upon the ability of the other ingredients in the composition to distribute and retain the active ingredient over the mucosal membranes within the nasal cavity and, if appropriate, the nasolacrimal canal. The distribution and retention properties of a pharmaceutical composition over a mucosal membrane are hereafter generally referred to as the “mucoadhesive” properties of the composition.

The mucoadhesive properties of a pharmaceutical composition are of great interest to pharmaceutical formulators and drug delivery scientists, as they can contribute to the controlled release of the active ingredient on the mucosal membranes.

The term “adhesion” refers to the intermolecular forces which hold matter together, particularly contiguous surfaces of neighboring media. An “adhesive” is a substance capable of joining two materials by adhesion. “Bioadhesion” implies that at least one of the two materials is of biological origin. When one surface is the adherent mucus layer covering the mucosal epithelia, the term “mucoadhesion” is used. Mucoadhesion is a particular form of bioadhesion; mucoadhesive materials may be useful to provide prolonged adhesion, and thus improved efficacy, of pharmaceutically active ingredients on mucosal tissue.

Hydroxypropyl methylcellulose (HPMC) is used in aqueous pharmaceutical compositions for the nasal administration of various pharmaceutically active ingredients. Further details related to HPMC may be found in U.S. Patent Application Pub. No. 2007/0104791, incorporated by reference herein.

Capsaicinoids are irritants, and produce a sensation of burning or hotness when they come in contact with human tissue. Capsaicinoids chemically interact with sensory neurons by binding to thermoreceptor nerve endings inside the body, or to receptors in the skin. These receptors may be stimulated with heat or physical abrasion, causing chemical signals to pass through the cell membrane and into the cell which causes the neuron to generate its own signal to the brain. The capsaicinoid molecules induce the same effect of producing a burning sensation, but not a chemical burn, that heat or physical abrasion achieves by binding to the thermoreceptor nerve endings.

Capsaicinoids have many uses, such as a heat additive for foods, topical ointments, and non-lethal weapons. They are used in foods to add spice or “heat,” such as in hot sauce or salsa. Capsaicinoids are also used as a topical ointment to relieve minor aches and pains in muscles and joints, such as symptoms associated with arthritis. They are also used as an ingredient in the non-lethal weapon commonly known as “pepper spray,” which when sprayed into the eyes or onto the skin is painful to the recipient. Further details related to capsaicinoids may be found in U.S. Patent Application Pub. No. 2008/0242741, also incorporated by reference herein.

Unlike many other all-natural and homeopathic products, the chemical structure of capsaicin (a type of capsaicinoid) and its mechanisms of action in animals and humans have been well characterized. The genes encoding their production have also been recently identified. Capsaicin is a member of the vanilloid family and binds to a receptor called the vanilloid receptor subtype 1 (VR1).

The burning and painful sensations associated with capsaicin result from its chemical interaction with sensory neurons. They interact with the VR1 ion channel-type receptors, which then permit cations to pass through the cell membrane and into the cell. The resulting depolarization of the neuron stimulates it to signal the brain.

More recently, the VR1 ion channel receptor has been shown to be a member of the “superfamily” of TRP ion channels, and is now referred to as TRPV1. There are a number of different TRP ion channels that have been shown to be sensitive to different ranges of temperature and probably are responsible for the human range of temperature sensation. Thus, capsaicin does not actually cause a chemical burn, or indeed any damage to tissue at all; it causes only the sensation of one.

Based on this rationale, capsaicin has been proposed as a means against chronic pain. With exposure to capsaicin, neurons are overwhelmed by the ion influx and are unable to report pain for an extended period of time with a blockade of neurogenic inflammation ensuing. If capsaicin is removed, the neurons recover.

Capsaicin reduces pain by selectively activating polymodal nociceptive neurons. Repetitive administrations of capsaicin produce a desensitization and an inactivation of sensory neurons. Several mechanisms are involved, including: receptor inactivation, block of voltage activated calcium channels, intracellular accumulation of ions leading to osmotic changes, and activation of proteolytic enzyme processes. Systemic and topical capsaicin produce a reversible anti-nociceptive and anti-inflammatory action after an initial undesirable analgesic effect.

Apart from use in the prevention of pain, capsaicin has also been used as a dietary supplement and pest repellant, as well as in cancer treatment and the treatment of autoimmune diabetes. It is also part of standard procedure in cough threshold measurement. Prior methods have been attempted to increase the tolerability (reduce burning) and to prolong the efficacy of the solution. Decreasing the amount of Capsicum (the fruit of most species of Capsicum contains capsaicin) increased tolerability, but appeared not to provide the needed efficacy.

The present invention relates to a method of preparing a homeopathic composition using capsaicin as an active ingredient (e.g., CLEARCAP® Super Soluble Capsicum), along with a mucoadhesive material that prolongs the adherence of the capsaicin to the nasal mucosa. The composition, which may be applied intranasally, provides relief for allergies (allergic rhinitis) as well as relief from sinus congestion, headaches (migraine, tension, cluster and premenstrual), colds (congestion, runny nose, post nasal drip) and flu symptoms. The combination of materials of the present invention has heretofore not been made, and provides surprisingly effective outcomes, as described below.

### SUMMARY OF THE INVENTION

The present invention relates to a method of preparing an intranasal composition comprising from about 0.0010-0.0040% by weight capsaicin, from about 0.50-0.90% by weight hydroxypropyl methylcellulose, from about 0.010-0.080% by weight n-alkyl dimethyl benzyl ammonium chloride (Quat), and optionally various moisturizing and irrigation agents, wherein the method comprises dissolving the above components in water to form an aqueous solution; and filtering the aqueous solution to form an intranasal composition.

The present invention further relates to an intranasal drug delivery composition, which comprises from about 0.0010-0.0040% by weight capsaicin, from about 0.50-0.90% by weight hydroxypropyl methylcellulose, from about 0.010-0.080% by weight n-alkyl dimethyl benzyl ammonium chloride (Quat), and optionally various moisturizing and irrigation agents. The capsaicin is commercially available as CLEARCAP® Super Soluble Capsicum from Kalsec, Inc.; the HPMC is commercially available as METHOCEL™ from Dow Chemical Co.; and the n-alkyl dimethyl benzyl ammonium chloride is commercially available as STEPANQUAT® from Stepan Company. The present invention further relates to a method to increase tolerability and reduce burning from capsaicin when applied to nasal mucosa in the form of an intranasal composition, which comprises combining capsaicin with the materials as noted above.

### DETAILED DESCRIPTION OF THE INVENTION

The present invention relates to a method of preparing a homeopathic composition using capsaicin as an active ingredient (e.g., CLEARCAP® Super Soluble Capsicum), along with a mucoadhesive material that prolongs the adherence of the capsaicin to the nasal mucosa. The composition, which is applied intranasally, provides relief for allergies (allergic rhinitis) as well as relief from sinus congestion, headaches (migraine, tension, cluster and premenstrual), colds (congestion, runny nose, post nasal drip) and flu symptoms.

In preferred embodiments, the present invention may include the following more specific components (in weight percent): about 0.0028% capsaicin, about 0.75% HPMC, about 0.010% Quat, and about 1.1% moistening and irrigation components. In further preferred embodiments, the hydroxypropyl methylcellulose has a viscosity of from about 2500 to 5500 cps (mPa·s), and the moisturizing and irrigation agents are selected from eucalyptus oil, aloe vera whole leaf gel and refined sea salt. The intranasal composition may also further comprise ascorbic acid or related substances.

In the manufacture of the present invention, the following procedures may be followed to create an exemplary composition, starting with the preparation of a solution (designated as “Solution A”) and ending with a final composition. Such manufacturing criteria are discussed below.

### I. Preparation of Solution A

In the preparation of Solution A, an HPMC dispersion is first created. To 1000 pounds of DI (distilled) or USP (purified) water at about 200° C., the addition of 19.4 pounds of sodium chloride is made. This is followed by heating to about 65° C. and stirring at 60 r.p.m. until complete dissolution is attained (about 20 minutes). When a temperature of about 65° C. is reached, approximately 15.0 pounds of METHOCEL E4M is added in portions (about 2 ounces each) at 60 r.p.m., followed by stirring for 30 minutes after the last added portion.

Following the above steps, heating is cancelled and cooling commenced to about 200° C. Approximately 500 pounds of purified water at a temperature of about 200° C. is then added to the METHOCEL E4M Dispersion, followed by stirring at 40 r.p.m until a temperature of about 200° C. is reached (but not less than 150 minutes) to obtain Solution A.

### II. Preparation of Solution B

In a suitable mixing device, 25 pounds of purified water at 200° C. is added, followed by the addition of 0.4 pounds ascorbic acid USP and stirring at 200 r.p.m. until full dissolution (not less than 10 minutes) is reached. This is followed by the addition of 0.2 pounds STEPANQUAT 50 NF, 0.056 pounds CLEARCAP® Super Soluble Capsicum, 0.4 pounds rosemary glycolic extract and 1 pound aloe vera whole leaf gel 2× under constant stirring at 200 rpm, to obtain Solution B.

### III. Preparation of Solution C

Solution B is added to Solution A at a temperature of about 20° C. while continuously stirring at 40 r.p.m. for 10 minutes. Following this step, 0.8 pounds eucalyptus oil is added, and the materials stirred at 40 r.p.m. for an additional 30 minutes. The mixture is then completed to 2000 pounds with purified water and stirred at 40 r.p.m. for 30 minutes to obtain Solution C, which is filtered through a 75 micron filter to obtain a final composition, which appears as a clear liquid and with a pH of from about 3.4-3.9, and a refractive index of from about 1.330 to 1.336.

An experimental study of the nasal application of the intranasal composition of the present invention demonstrated significant symptom relief in patients with allergic rhinitis. A total nasal symptom score (TNSS), the sum of the subjects' daily morning and evening individual nasal symptoms, was generated, with patients receiving treatments of the final composition as described above, in a double-blind, randomized cross-over comparison with and without the hydroxypropyl methylcellulose (HPMC) component. While both treatments resulted in a significantly decreased TNSS, it was found that the composition of the present invention (i.e., including the HPMC component) allowed for a prolonged duration of treatment being tolerated, with less frequent dosing required. It is speculated that this effect does not depend on the duration of the contact with the mucosa, but upon triggering of local neuronal networks in the nose.

The capsaicin in the intranasal composition of the present invention blocks C-fiber conduction, inactivates neuropeptide release from peripheral nerve endings, causes neuropeptide depletion and reduction of neurogenic inflammation and as a result, amelioration of the bothersome nasal symptoms associated with allergic rhinitis.

While the present invention has been described with respect to particular embodiments thereof, it is apparent that numerous other forms and modifications of the invention will be obvious to those skilled in the art. The appended claims and the present invention generally should be construed to cover all such obvious forms and modifications which are within the true spirit and scope of the present invention.

1. A method for the preparation of an intranasal composition consisting essentially of 0.0010-0.0040% by weight capsaicin, 0.50-0.90% by weight hydroxypropyl methylcellulose, 0.010-0.080% by weight n-alkyl dimethyl benzyl ammonium chloride (Quat), and various moisturizing and irrigation agents; wherein the method comprises dissolving the above components in water to form an aqueous solution, and filtering the aqueous solution to form an intranasal composition.

2. The method as recited in claim 1, wherein the hydroxypropyl methylcellulose has a viscosity of from about 2500 to 5500 cps (mPa·s).

3. The method as recited in claim 1, wherein the moisturizing and irrigation agents are selected from eucalyptus oil, aloe vera whole leaf gel and refined sea salt.

4. The method as recited in claim 1, wherein the intranasal composition further comprises ascorbic acid.

5. The method as recited in claim 1, comprising about 0.0028% by weight capsaicin.

6. The method as recited in claim 1, comprising about 0.75% by weight hydroxypropyl methylcellulose.

7. The method as recited in claim 1, comprising about 0.010% by weight n-alkyl dimethyl benzyl ammonium chloride (Quat).

8. An intranasal composition comprising from about 0.0010-0.0040% by weight capsaicin, from about 0.50-0.90% by weight hydroxypropyl methylcellulose, from about 0.010-0.080% by weight n-alkyl dimethyl benzyl ammonium chloride (Quat), and various moisturizing and irrigation agents.

9. The composition as recited in claim 8, wherein the hydroxypropyl methylcellulose has a viscosity of from about 2500 to 5500 cps (mPa·s).

10. The composition as recited in claim 8, wherein the moisturizing and irrigation agents are selected from eucalyptus oil, aloe vera whole leaf gel and refined sea salt.

11. The composition as recited in claim 8, wherein the intranasal composition further comprises ascorbic acid.

12. The composition as recited in claim 8, comprising about 0.0028% by weight capsaicin.

13. The composition as recited in claim 8, comprising about 0.75% by weight hydroxypropyl methylcellulose.

14. The composition as recited in claim 8, comprising about 0.010% by weight n-alkyl dimethyl benzyl ammonium chloride (Quat).

15. A method to increase tolerability and reduce burning from capsaicin when applied to nasal mucosa in the form of an intranasal composition, which comprises applying to said nasal mucosa an intranasal composition comprising from about 0.0010-0.0040% by weight capsaicin, from about 0.50-0.90% by weight hydroxypropyl methylcellulose, from about 0.010-0.080% by weight n-alkyl dimethyl benzyl ammonium chloride (Quat), and various moisturizing and irrigation agents.

16. The method as recited in claim 15, wherein the hydroxypropyl methyl cellulose has a viscosity of from about 2500 to 5500 cps (mPa·s).

17. The method as recited in claim 15, wherein the moisturizing and irrigation agents are selected from eucalyptus oil, aloe vera whole leaf gel and refined sea salt.

18. The method as recited in claim 15, wherein the intranasal composition further comprises ascorbic acid.

19. The method as recited in claim 15, comprising about 0.0028% by weight capsaicin.

20. The method as recited in claim 15, comprising about 0.75% by weight hydroxypropyl methylcellulose.

21. The method as recited in claim 15, comprising about 0.010% by weight n-alkyl dimethyl benzyl ammonium chloride (Quat).

22. A method for the preparation of an intranasal composition consisting essentially of 0.0028% by weight capsaicin, 0.75% by weight hydroxypropyl methylcellulose, 0.010% by weight n-alkyl dimethyl benzyl ammonium chloride (Quat), and various moisturizing and irrigation agents; wherein the method comprises dissolving the above components in water to form an aqueous solution, and filtering the aqueous solution to form an intranasal composition.

23. The method as recited in claim 22, wherein the hydroxypropyl methylcellulose has a viscosity of from about 2500 to 5500 cps (mPa·s).

24. The method as recited in claim 22, wherein the moisturizing and irrigation agents are selected from eucalyptus oil, aloe vera whole leaf gel and refined sea salt.

25. The method as recited in claim 22, wherein the intranasal composition further includes ascorbic acid.
